(19) United States
(12) Patent Application Publication
(10) Pub. No.: US 2018/0206960 A1
(43) Pub. Date: Jul. 26, 2018

(54) DENTAL TREATMENT FILMS INCORPORATING UNMIXED REACTIVE INGREDIENTS

(71) Applicant: CAO GROUP, INC., West Jordan, UT (US)

(72) Inventor: Steven D. Jensen, West Jordan, UT (US)

(21) Appl. No.: 15/744,904

(22) PCT Filed: Jul. 21, 2016
(86) PCT No.: PCT/US16/43329

§ 371 Date: Jan. 15, 2018

Related U.S. Application Data
(60) Provisional application No. 62/195,501, filed on Jul. 22, 2015.

(51) Int. Cl. A61C 19/06; A61K 9/00; A61K 9/70

(52) U.S. Cl. CPC A61C 19/063; A61K 9/16; A61K 9/7007; A61K 9/0063

(57) ABSTRACT

A two-part treatment film may include a barrier layer, a gelatinous composition layer, and at least one active ingredient adhered to the gelatinous composition layer. The barrier layer may be a flexible film that is insoluble in water. In some embodiments, the at least one active ingredient may be in the form of dried particles. In other embodiments, the at least one active ingredient may be in the form of microspheres filled with an active ingredient.

### CROSS-REFERENCES TO RELATED APPLICATIONS

This application claims the benefit of PCT Application No. PCT/US2016/043329, filed Jul. 21, 2016, which is a non-provisional of U.S. Provisional Patent Application No. 62/195,501 filed on Jul. 22, 2015, each of which is hereby incorporated by reference in its entirety.

### TECHNICAL FIELD OF THE INVENTION

The present invention comprises a two-part delivery modality in order to successfully deliver an intended treatment.

### BACKGROUND OF THE INVENTION

Usually, delivering a two-part treatment requires a device that provides separate chambers where chemical components can be stored away from each other until the time of use. Some chemicals are unstable when mixed together; as such they have no appreciable shelf life and therefore cannot be made into a viable commercial product. Other treatments require a chemical reaction to occur at the time of delivery. two-part treatment systems are designed to solve these problems; they are designed to keep unstable and reactive components separate until the time they are delivered, whereupon they are mixed and placed at the treatment site when needed.

An example of such a device is a double-barreled syringe with a motionless mixing tip. In general, a chemical composition A is loaded into one barrel and a chemical composition B is loaded into the other barrel. In this way, composition A and B are stored away from each other until it becomes necessary to deliver the combined chemical composition “C” upon mixing and delivery (A+B=C). The components are delivered by pressing down upon the syringe plunger expressing components A and B into the motionless mixing tip wherein it mixes the components together resulting in composition C that is expressed out of the tip nozzle and onto the desired treatment site.

The double barrel syringe has been a common device in the medical and dental field that effectively delivers two-part chemical compositions for a variety of treatments. In recent years, the invention of film or strip type products has been utilized as a means to deliver one-part chemical compositions to the oral cavity. Film and Strip type products comprise a barrier layer that is made from a thin insoluble plastic sheet and a chemical composition layer that is placed on one surface of the plastic sheet creating a finished product. In practice, the chemical composition layer is placed against the teeth or tissue and adhered into place; the barrier layer is by default positioned on the opposing side of the treatment site in order to protect the chemical composition from being washed away by saliva. Film and strip type products are efficient devices to deliver homogenous composition systems; patients prefer films and strips to the old syringe methods of delivery. Films and strips are easier to use and more convenient for the patient; these are important factors because it encourages patients to comply with mandatory treatment regimes.

### SUMMARY OF THE INVENTION

Embodiments of the present invention provide films and strips that are capable of delivering multiple/heterogeneous chemical compositions to a treatment surface simply by applying the strip/film.

Embodiments of the present invention utilize a barrier layer that is comprised of a polymeric sheet. An ideal barrier layer may be thin, flexible and insoluble in water. The barrier layer may constitute the 1^{st} layer in the manufacture of a treatment film; it is on one surface of the barrier layer that the 2^{nd} treatment layer may be adhered. Examples of barrier layer materials include but are not limited to: parafilm, polyethylene sheet, polypropylene sheet, PVC sheet, polyurethane sheet, nylon sheet, PVA sheet, and the like.

### BRIEF DESCRIPTION OF THE DRAWINGS

In order to describe the manner in which the above-recited and other advantages and features of the invention can be obtained, a more particular description of the invention briefly described above will be rendered by reference to specific example embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings.

FIG. 1 shows a side view of a two-part chemical composition strip/film according to an embodiment of the present invention.

FIG. 2 shows a side view of a two-part chemical composition strip/film comprising hollow micro-spheres that are filled with active ingredients that comprise the second half of the two-part system according to an additional embodiment of the present invention.

FIG. 3 shows a side view of a two-part chemical composition strip/film that comprises hollow micro-spheres are mixed and embedded within another layer according to an additional embodiment of the present invention.

### DETAILED DESCRIPTION

Embodiments of the present invention utilize a barrier layer that is comprised of a polymeric sheet. An ideal barrier layer may be thin, flexible and insoluble in water. The barrier layer may constitute a first layer in the manufacture of a treatment film; it is on one surface of the barrier layer that a second treatment layer is adhered. Examples of barrier layer materials include but are not limited to: parafilm, polyethylene sheet, polypropylene sheet, PVC sheet, polyurethane sheet, nylon sheet, PVA sheet, and the like.
One embodiment of the present invention comprises three layers:
A. Barrier layer
B. Gelatinous composition layer
C. Surface adhered active ingredient particulates/granules

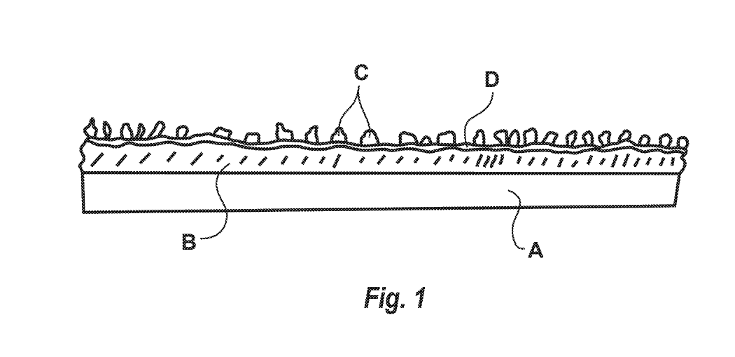

FIG. 1 shows an embodiment of a two-part chemical composition strip/film. The film is comprised of a barrier layer A (The first layer). The barrier layer A may be comprised of a thin polymeric sheet that is flexible and water insoluble. On one surface of the barrier layer A is applied a gelatinous composition layer B (The second layer). The gelatinous composition layer B may be comprised of a water-based gellant/thickener homogenously mixed with one portion of the active ingredients that constitutes one part of a two-part system. This homogenous mixed gel may then be applied onto the barrier layer A as a thin layer and then adhered into place by drying the gel into the gelatinous layer B. When dried appropriately, the gelatinous layer B will have the flexibility and consistency of the common confection known as “gummy worms”. The process of drying produces a semi-dehydrated layer on the surface of the gelatinous layer B; this physical phenomenon is called a surface skin D. The surface skin D is a dehydrated skin that forms as a result of drying; the surface skin D protects the more hydrated portions of the composition directly beneath the skin's surface. It is within/on the surface skin D that the dry ingredients of an Active Ingredient particulates layer C are adhered/embedded (The third layer). The active ingredient particulates are either physically embedded within the skin's surface or adhered by the natural tackiness of the surface skin D. (Steam or an organic wetting agent could be utilized to temporarily create or increase the tackiness of the surface skin D just before applying the active ingredient particulates layer C). The dry ingredients of the active ingredient particulates layer C are applied similar to applying salt from a saltshaker. The dry ingredients of the active ingredient particulates layer C are not intended to cover the entire surface of the gelatinous composition layer B, it is intended to leave a portion of the gelatinous composition layer B uncovered in order for the strip/film to adhere to the teeth or tissue of the treatment site. The active ingredient particulates that comprise layer C constitute the second half of the two-part system. By design, there is insufficient water in the surface skin D to fully activate the dry active ingredient granules that are adhered or imbedded within or on its surface. Therefore, the active ingredient particulates layer C and the gelatinous composition layer B comprise adhered but separate compositions of a two-part system. By design, the particulates in the active ingredient particulates layer C are readily dissolvable when introduced with water or saliva of the oral cavity.

The resulting film/strip is activated when it is applied to a moist treatment area within the oral cavity. Upon application, the saliva dissolves the dry ingredients of the active ingredient particulates layer C and at the same time hydrates the surface skin layer D; this allows the separated components to begin mixing after being placed adjacent the treatment site. The patient may aid the mixing of the layers while hand applying the strip/film to the teeth or tissue; the pressure applied against the barrier layer A will produce forces that squish the layers together that further mix the two-part system. Furthermore, the active forces within the oral cavity will aid the mixing of the layers while the regime continues, i.e. increased temperature to about 37 degrees C., mild occlusal forces, and continuous interactions between cheeks and teeth. Upon mixing, the two-part system becomes activated delivering the desired reaction chemicals or shelf unstable compounds to the treatment site in a convenient manner.

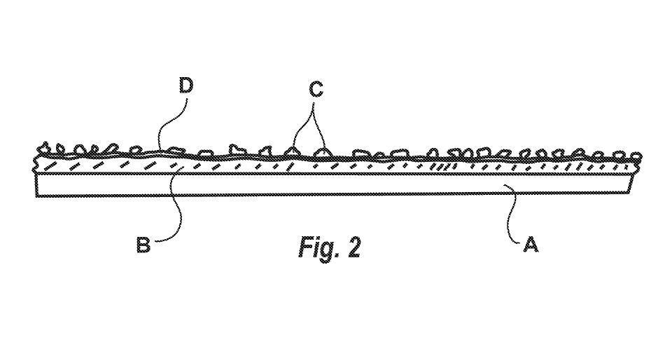

Another embodiment of the present invention is shown in FIG. 2. The strip/film of FIG. 2 comprises the same layers as FIG. 1 as well as their descriptions with the exception of Layer C. In FIG. 2, a layer C is comprised of hollow micro-spheres that are filled with the active ingredients that comprise the second half of the two-part system. The micro-spheres comprise spherical hollow shells that deliver their internal contents by multiple means:

By design, the hollow shells of the layer C can be made of materials that readily dissolve in water or saliva; thus the internal active ingredients are released when placed against a wet treatment surface and the outer shell is readily dissolved. The released compounds then mix with the active ingredients contained within a layer “B”. Upon mixing, the two-part system becomes activated delivering the desired reaction chemicals or shelf unstable compounds to the treatment site in a convenient manner.

By design, the micro-spheres of the layer C can be made of materials that have a low crush strength. When the layer C is placed against the treatment site the force of application causes the micro-spheres to become ruptured releasing their contents to readily mix with the layer “B”. Upon mixing, the two-part system becomes activated delivering the desired reaction chemicals or shelf unstable compounds to the treatment site in a convenient manner.

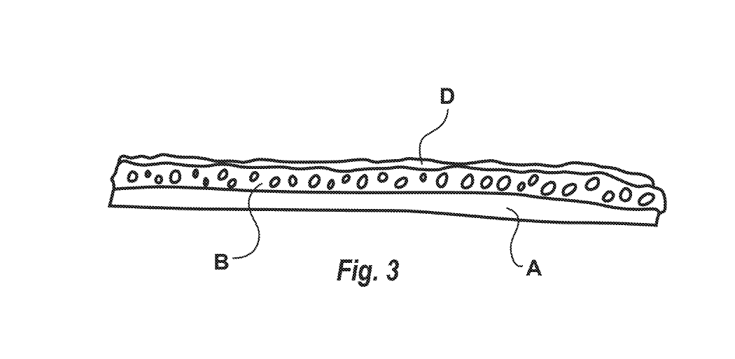

Another embodiment of the present invention is shown in FIG. 3. FIG. 3 includes all the identical layers of FIG. 2 with the exception of layer C, which is missing. In FIG. 3 the hollow micro-spheres of the layer C of FIG. 2 are instead mixed and embedded within a layer B. By design, the micro-spheres that are mixed and imbedded within the layer B are made of materials that have a low crush strength. When the layer B is placed against the treatment site the force of application causes the micro-spheres to become ruptured releasing their contents to readily mix with the gelatinous portion of the layer B. Upon mixing, the two-part system becomes activated delivering the desired reaction chemicals or shelf unstable compounds to the treatment site in a convenient manner.

Upon practical application of the embodiments of the present invention many other embodiments that fall under the scope of this patent become evident.

What is claimed is:

1. A two-part treatment film comprising:
a barrier layer;
a gelatinous composition layer; and
at least one active ingredient adhered to the gelatinous composition layer.

2. The two-part treatment film of claim 1, wherein the barrier layer is insoluble in water.

3. The two-part treatment film of claim 2, wherein the barrier layer comprises a flexible film.

4. The two-part treatment film of claim 1, wherein the gelatinous composition layer comprises a dehydrated surface skin.

5. The two-part treatment film of claim 1, wherein the at least one active ingredient comprises dried particles.

6. The two-part treatment film of claim 1, wherein the at least one active ingredient comprises microspheres filled with an active ingredient.
